(19) United States
(12) Patent Application Publication
(10) Pub. No.: US 2018/0042518 A1
(43) Pub. Date: Feb. 15, 2018

(54) POSITION SENSOR FOR A MEDICAL PROBE

(71) Applicant: Cardiac Pacemakers, Inc., St. Paul, MN (US)

(72) Inventors: Angelo Fruci, Mahtomedi, MN (US); Daniel J. Foster, Lino Lakes, MN (US); Doug E. Giwoyna, Harris, MN (US); James E. Blood, Shoreview, MN (US); David R. Wulfman, Minneapolis, MN (US); Michael A. Felling, Stillwater, MN (US)

(21) Appl. No.: 15/674,921

(22) Filed: Aug. 11, 2017

Related U.S. Application Data
(60) Provisional application No. 62/374,559, filed on Aug. 12, 2016.

(51) Int. Cl. A61B 5/06; A61B 18/14

(52) U.S. Cl. CPC A61B 5/062; A61B 5/063; A61B 18/1492; A61B 5/02028

(57) ABSTRACT

A position sensor assembly includes a base member having a proximal portion, a distal portion, and an intermediate portion disposed therebetween and having a twisted configuration such that the proximal and distal portions are oriented in mutually orthogonal planes. At least first and second magnetic field sensors each including at least one magnetic field sensing element are disposed, respectively, on the proximal and distal portions of the base member. The base member further includes a first base member element defining the proximal portion of the base member, and a second base member element defining the distal portion of the base member, the first and second base member elements being electrically and mechanically connected at a joint.

### CROSS-REFERENCE TO RELATED APPLICATION

This application claims priority to Provisional Application No. 62/374,559, filed Aug. 12, 2016, which is herein incorporated by reference in its entirety.

### TECHNICAL FIELD

The present invention relates to medical devices and methods for delivering a therapy to the body. More specifically, the invention relates to devices and methods for detecting a position of the device within the body.

### BACKGROUND

Cardiac arrhythmia and/or other cardiac pathology contributing to abnormal heart function may originate in cardiac cellular tissue. One technique that may be utilized to treat the arrhythmia and/or cardiac pathology may include ablation of tissue substrates contributing to the arrhythmia and/or cardiac pathology. Ablation by heat, chemicals or other means of creating a lesion in the tissue substrate may isolate diseased tissue from normal heart circuits. In some instances, electrophysiology therapy may involve locating tissue contributing to the arrhythmia and/or cardiac pathology using a mapping and/or diagnosing catheter and then using an ablation electrode to destroy and/or isolate the diseased tissue.

Prior to performing an ablation procedure, a physician and/or clinician may utilize specialized mapping and/or diagnostic catheters to precisely locate tissue contributing and/or causing an arrhythmia or other cardiac pathology. It is often desirable to precisely locate the targeted tissue prior to performing an ablation procedure in order to effectively alleviate and/or eliminate the arrhythmia and/or cardiac pathology. Further, precise targeting of the tissue may prevent or reduce the likelihood that healthy tissue (located proximate the targeted tissue) is damaged.

Several methods and/or techniques may be employed to precisely locate targeted tissue where an ablation or other therapeutic procedure may be performed. An example method may include utilizing an ablation, mapping and/or diagnostic catheter to determine how close the catheter is to targeted tissue. Further, the ablation, mapping and/or diagnostic catheter may include one or more sensing electrodes located on a distal portion of the catheter. The electrodes may sense, measure and/or provide a processor with information relating to electrical characteristics of the cardiac tissue and surrounding media. Using the sensed and/or measured information, the processor may be able to correlate the spatial location of the distal portion of the catheter to the cardiac tissue. For example, electrodes may sense the impedance, resistance, voltage potential, etc. of the cardiac tissue and/or surrounding media and determine how far a distal portion of a diagnostic and/or ablation catheter is to cardiac tissue.

To locate the catheter and electrodes within the body, the catheter may include a position sensor configured to provide an indication of a location within a multidimensional magnetic field.

### SUMMARY

A position sensor assembly comprising a base member, a first magnetic field sensor and a second magnetic field sensor. The base member has a substantially linear longitudinal axis, a proximal portion oriented in a first plane along the longitudinal axis, a distal portion oriented in a second plane along the longitudinal axis, the second plane being substantially orthogonal to the first plane, and an intermediate portion extending between the proximal and distal portions in a twisted configuration to operate as a transition between the proximal and distal portions. The base member includes a first base member element defining the proximal portion, and a second base member element defining the distal portion, and further wherein the first and second base member elements are mechanically and electrically coupled together at a joint. The first magnetic field sensor is disposed on the proximal portion of base member, the first magnetic field sensor including a first magnetic field sensing element, the first magnetic field sensor being oriented on the proximal portion of the base member so that the first magnetic field sensing element has a sensitivity to a component of the multi-dimensional magnetic field along a first axis. The second magnetic field sensor is disposed on the distal portion of base member, the second magnetic field sensor including a second magnetic field sensing element, the second magnetic field sensor oriented on the distal portion of the base member so that the second magnetic field sensing element has a sensitivity to a component of the multi-dimensional magnetic field along a second axis.

In another embodiment, a medical probe comprising a flexible catheter body having a distal end, an active element coupled to the distal end of the flexible catheter body, and a position sensor assembly carried by the catheter body proximate the active element. The position sensor assembly comprises a base member, a first magnetic field sensor and a second magnetic field sensor. The base member has a substantially linear longitudinal axis, a proximal portion oriented in a first plane along the longitudinal axis, a distal portion oriented in a second plane along the longitudinal axis, the second plane being substantially orthogonal to the first plane, and an intermediate portion extending between the proximal and distal portions in a twisted configuration to operate as a transition between the proximal and distal portions. The base member includes a first base member element defining the proximal portion, and a second base member element defining the distal portion, and further wherein the first and second base member elements are mechanically and electrically coupled together at a joint. The first magnetic field sensor is disposed on the proximal portion of base member, the first magnetic field sensor including a first magnetic field sensing element, the first magnetic field sensor being oriented on the proximal portion of the base member so that the first magnetic field sensing element has a sensitivity to a component of the multi-dimensional magnetic field along a first axis. The second magnetic field sensor is disposed on the distal portion of base member, the second magnetic field sensor including a second magnetic field sensing element, the second magnetic field sensor oriented on the distal portion of the base member so that the second magnetic field sensing element has a sensitivity to a component of the multi-dimensional magnetic field along a second axis.

In another embodiment, a medical system comprising a medical probe, a magnetic field generator and a processor. The medical probe comprises a flexible catheter body having a distal end, an active element coupled to the distal end of the flexible catheter body, and a position sensor assembly carried by the catheter body proximate the active element. The position sensor assembly comprises a base member, a first magnetic field sensor and a second magnetic field sensor. The base member has a substantially linear longitudinal axis, a proximal portion oriented in a first plane along the longitudinal axis, a distal portion oriented in a second plane along the longitudinal axis, the second plane being substantially orthogonal to the first plane, and an intermediate portion extending between the proximal and distal portions in a twisted configuration to operate as a transition between the proximal and distal portions. The base member includes a first base member element defining the proximal portion, and a second base member element defining the distal portion, and further wherein the first and second base member elements are mechanically and electrically coupled together at a joint. The first magnetic field sensor is disposed on the proximal portion of base member, the first magnetic field sensor including a first magnetic field sensing element, the first magnetic field sensor being oriented on the proximal portion of the base member so that the first magnetic field sensing element has a sensitivity to a component of the multi-dimensional magnetic field along a first axis. The second magnetic field sensor is disposed on the distal portion of base member, the second magnetic field sensor including a second magnetic field sensing element, the second magnetic field sensor oriented on the distal portion of the base member so that the second magnetic field sensing element has a sensitivity to a component of the multi-dimensional magnetic field along a second axis. The magnetic field generator is configured to generate a multi-dimensional magnetic field in a volume including the medical probe and a patient. The processor is operable to receive outputs from the magnetic field sensors to determine a position of the position sensor assembly within the volume.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 shows an exemplary position sensing system for determining the location of a position sensor within a multidimensional magnetic field.

FIGS. 2-6 show various embodiments of a position sensor assembly configured for providing an indication of a location within a multidimensional magnetic field.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

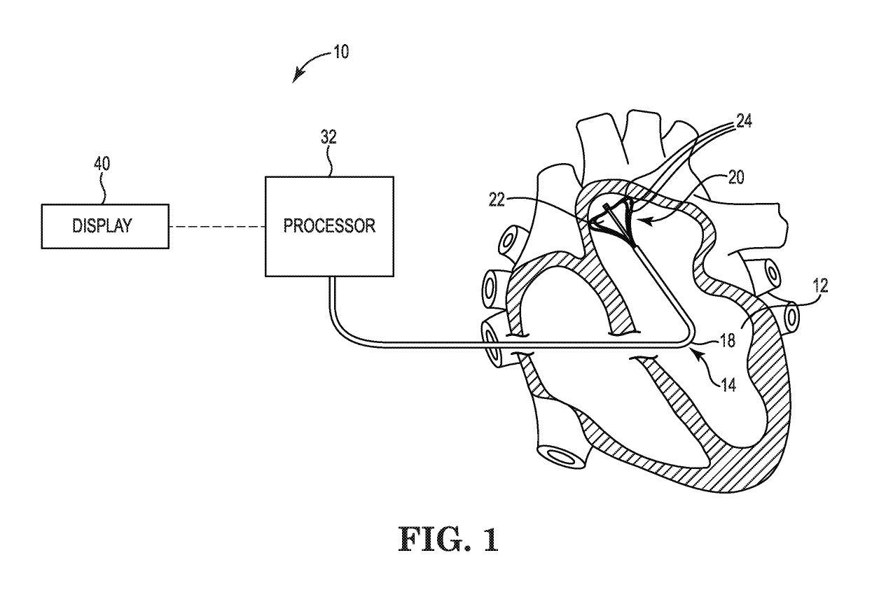

FIG. 1 is a schematic view of a medical system 10 for accessing a targeted tissue region in the body of a patient for diagnostic and/or therapeutic purposes. FIG. 1 generally shows the system 10 deployed in a region of the heart. For example, system 10 may be deployed in any chamber of the heart, such as the left atrium, left ventricle, right atrium, or right ventricle, another region of the cardiovascular system, or other anatomical region. The system 10 further is configured to be used in conjunction with a magnetic position tracking system (not shown) that includes a magnetic field generator for producing a multidimensional magnetic field in a predetermined working volume in which the body of the patient is located. The field generator is positioned at known spatial coordinates with respect to the body of the patient. Except as otherwise specifically discussed herein, the magnetic position tracking system may be of any design or configuration, now known or later developed, suitable for use with a medical system such as the system 10.

In the illustrated embodiment, the system 10 includes a mapping catheter or probe 14. Each probe 14 may be separately introduced into the selected heart region 12 through a vein or artery (e.g., the femoral vein or artery) using a suitable percutaneous access technique. Alternatively, the system 10 may include one or more probes that have both mapping and therapeutic capabilities (e.g., a radiofrequency (RF) ablation catheter having one or more sensing electrodes for acquiring electrical signals from the patient's heart).

In the illustrated embodiment, the mapping probe 14 may include flexible catheter body 18, the distal end of which carries a three-dimensional multiple electrode structure 20. In the illustrated embodiment, the electrode structure 20 takes the form of a basket formed from a plurality of splines together defining an open interior space 22, although other electrode structures could be used. The electrode structure 20 carries a plurality of mapping electrodes 24 each having an electrode location on a respective spline of the electrode structure 20 (for ease of illustration, the electrodes 24 are depicted only on a single spline in FIG. 1; it will be appreciated, however, that each spline may include one or more of the electrodes 24). Each mapping electrode 24 may be configured to sense electrical characteristics (e.g. voltage and/or impedance) in an adjacent anatomical region.

The electrodes 24 may be electrically coupled to the processor 32. A signal wire (not shown) may be electrically coupled to each electrode 24 on structure 20. The signal wires may extend through body 18 of probe 14 and electrically couple each electrode 24 to an input of the processor 32. Electrodes 24 may sense electrical characteristics correlated to an anatomical region adjacent to their physical location within the heart. The sensed cardiac electrical characteristic (e.g., voltage, impedance, etc.) may be processed by the processor 32 to assist a user, for example a physician, by generating processed output—e.g. an anatomical map (e.g., 3D map of heart chamber)—to identify one or more sites within the heart appropriate for a diagnostic and/or treatment procedure, such as an ablation procedure.

The processor 32 may include dedicated circuitry (e.g., discrete logic elements and one or more microcontrollers; application-specific integrated circuits (ASICs); or specially configured programmable devices, such as, for example, programmable logic devices (PLDs) or field programmable gate arrays (FPGAs)) for receiving and/or processing the acquired physiological activity. In some examples, processor 32 may include a general purpose microprocessor and/or a specialized microprocessor (e.g., a digital signal processor, or DSP, which may be optimized for processing activation signals) that executes instructions to receive, analyze and display information associated with the received physiological activity. In such examples, the processor 32 can include program instructions, which when executed, perform part of the signal processing. Program instructions can include, for example, firmware, microcode or application code that is executed by microprocessors or microcontrollers. The above-mentioned implementations are merely exemplary, and the reader will appreciate that processor 32 can take any suitable form for receiving electrical signals and processing the received electrical signals. The processor 32 further includes code for determining a location of the position sensor within the multidimensional magnetic field.

The mapping probe 14 including a position sensor (not shown in FIG. 1) carried by the catheter body 18 near the electrode structure 20. The position sensor is disposed at a location on the mapping probe that allows positioning of the position sensor within the anatomical structure (e.g., the heart) of interest.

The position sensor is communicatively coupled to the processor 32 by a wired or wireless communications path such that the processor 32 sends and receives various signals to and from the position sensor. As is known in the art, a position tracking system (not shown) including a magnetic field generator is configured to generate one or more magnetic fields that are sensed by the position sensor on the probe 14. The processor 32 is configured to process the output signals from the position sensor to resolve the location of the position sensor, and consequently, the distal portion of the probe 14, within the volume defined by the multi-dimensional magnetic field.

The processor 32 may output data to a suitable device, for example display device 40, which may display relevant information for a user. For example, the display device 40 may provide to the user a three-dimensional electroanatomical map of the cardiac chamber in which the mapping probe 14 is deployed. In some examples, device 40 is a display (e.g. a CRT, LED), or other type of display, or a printer. In addition, the processor 32 may generate position-identifying output for display on device 40 that aids the user in guiding an ablation electrode or other therapeutic device into contact with tissue at the site identified for ablation.

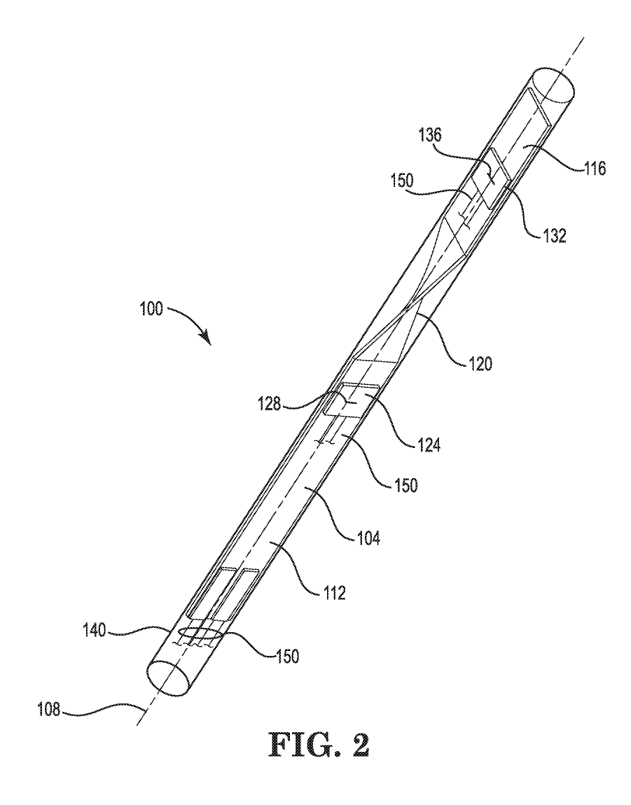

FIG. 2 is a schematic view of a position sensor assembly 100 that can be incorporated into the mapping probe 14 of the system 10, or in other embodiments, an alternative probe (e.g., an ablation catheter) for position tracking purposes. As shown in FIG. 2 the position sensor assembly 100 includes a base member 104 defining a substantially linear longitudinal axis 108. As further shown, the base member 104 includes a proximal portion 112, a distal portion 116, and an intermediate portion 120 between the proximal and distal portions. As shown, the proximal portion 112 is oriented in a first plane along the longitudinal axis 108, and the distal portion 116 is oriented in a second plane along the longitudinal axis 108, with the second plane being substantially orthogonal to the first plane.

As further shown, the intermediate portion 120 extends between the proximal and distal portions 112, 116 in a twisted configuration to operate as a transition between the proximal and distal portions 112, 116. In various embodiments, the intermediate portion 120 has a reduced stiffness with respect to the proximal and/or distal portions 112, 116, for example by reducing a thickness and/or a width of the transition zone base member. Alternatively, the intermediate portion 120 may be formed from a material having a lower stiffness than that of the proximal and/or distal portions 112, 116. In some embodiments, the axis of rotation about which the intermediate portion is twisted substantially corresponds to the longitudinal axis 108.

The position sensor assembly 100 further includes a first magnetic field sensor 124 having a first magnetic field sensing element 128, and a second magnetic field sensor 132 having a second magnetic field sensing element 136. As will be understood by those skilled in the art, the first and second magnetic field sensing elements 128, 136 are each configured to have a sensitivity to a component of a multi-dimensional magnetic field generated by an external field generator (as described previously) along a predetermined direction or axis.

In the illustrated embodiment, the first magnetic field sensor 124 is disposed on the proximal portion 112 of base member 104, and consequently, in the first plane. Additionally, the second magnetic field sensor 132 is disposed on the distal portion 116 of base member 104, and consequently, in the second plane, which as described and shown, is oriented generally orthogonal to the first plane.

In various embodiments, the position sensor assembly 100 may also include a third magnetic field sensor (not shown) having a third magnetic field sensing element substantially similar to the first and second magnetic field sensing elements. In such embodiments, the third magnetic field sensor may be disposed on the proximal portion 112 of the base member 104, but oriented thereon such that its axis of sensitivity is orthogonal to that of the first magnetic field sensing element 128. Alternatively, the third magnetic field sensor may be disposed on the distal portion 116 of the base member 104, but oriented thereon such that its axis of sensitivity is orthogonal to that of the second magnetic field sensing element 136.

In some embodiments, one or both of the first magnetic field sensor 124 and the second magnetic field sensor 132 may be a dual-axis sensor having two magnetic field sensing elements disposed on or within a single die, each magnetic field sensing element being oriented so that the axes of sensitivity of the respective magnetic field sensing elements are mutually orthogonal to one another. For example, in one embodiment, the first magnetic field sensor 124 may include both the first magnetic field sensing element 128 as well as the third magnetic field sensing element (not shown). Alternatively, in one embodiment, the second magnetic field sensor 132 may include both the second magnetic field sensing element 136 as well as the third magnetic field sensing element. Thus, the respective individual magnetic field sensing elements need not necessarily all be located on a separate die.

In the various embodiments, the position sensor assembly 100 is configured to sense the generated external magnetic fields and provide tracking signals indicating the location and orientation of the position sensor assembly 100 in up to six degrees of freedom (i.e., x, y, and z measurements, and pitch, yaw, and roll angles) when the first, second and third magnetic field sensors are present and oriented along mutually orthogonal axes.

In the various embodiments, the magnetic field sensors of the position sensor assembly 100 can include any magnetic field sensing technologies now known (e.g., anisotropic magneto-resistive (AMR) sensing elements, giant magneto-resistive (GMR) sensing elements, tunneling magneto-resistive (TMR) sensing elements, colossal magneto-resistive (CMR) sensing elements, extraordinary magneto-resistive (EMR) sensing elements, spin Hall sensing elements, and the like), or later developed.

As further shown, the position sensor assembly 100 includes a encapsulating element 140 (i.e., a housing) surrounding the base member 104 and the magnetic field field sensors disposed thereon. In embodiments, the encapsulating element 140 can be an epoxy material. Additionally, the position sensor assembly 100 includes conductors 150 for coupling the magnetic field sensors to electrical connection components (not shown) at or near the proximal portion 112 of the base member 104.

In various embodiments, position sensor assembly 100, particularly the base member 104 and the conductors 150 and associated electrical interconnects, may be constructed according to known, or later-developed, printed circuit board construction technologies. For example, the conductors 150 may be constructed as electrical traces formed on the body 104. Similarly, the body 104 may also be formed of any materials used for flexible printed circuit substrates. In other embodiments, the conductors 150 may be conductor wires that are separately bonded to the body 104 and the respective magnetic field sensors, either before or after forming the twist in the intermediate portion 120. As will be appreciated, the magnetic field sensors may discrete dies that are mounted to the base member 104 and electrically coupled to the conductors 150 according to known techniques.

In embodiments, the conductors 150 are disposed, at least within the intermediate portion 120, as near as possible to the longitudinal axis 108, which also constitutes the axis of rotation about which the intermediate portion 120 is twisted. In doing so, rotational and torsional stresses and strains on the conductors 150 disposed along the intermediate portion 120 of the base member 104 can be minimized.

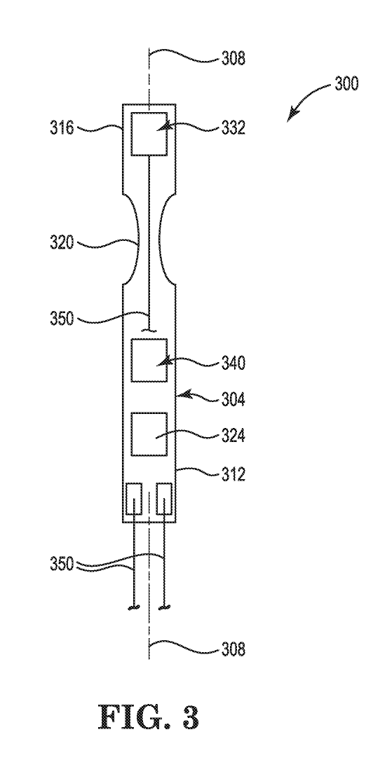

FIG. 3 is schematic view of a portion of a position sensor assembly 300 in an intermediate manufacturing state to illustrate the design according to one embodiment. Specifically, the position sensor assembly 300 includes a base member 304 defining a substantially linear longitudinal axis 308. As further shown, the base member 304 includes a proximal portion 312, a distal portion 316, and an intermediate portion 320 between the proximal and distal portions. The position sensor assembly 300 further includes a first magnetic field sensor 324 disposed on the proximal portion 312 of the base member 304, a second magnetic field sensor 332 disposed on the distal portion 316 of the base member 304, and a third magnetic field sensor 340 disposed on the proximal portion 312 at a different location than the first magnetic field sensor 324. As will be appreciated by the preceding discussion, in various embodiments, the third magnetic field sensor 340 may be instead located on the distal portion 316. Alternatively, in other embodiments, the first or second magnetic field sensor 324, 332 may be a dual-axis sensor having sensitivity in two mutually orthogonal axes, thus obviating the need for the third magnetic field sensor 340. As further shown, the position sensor assembly 300 includes a plurality of conductors 350 that operatively couple the respective magnetic field sensors to other electrical components (not shown).

In the particular embodiment illustrated, the intermediate portion 320 has an “hourglass” shape, such that it has concavely-curved outer edges resulting in a width at the middle of the intermediate portion 320 that is smaller than the width of either of the proximal or distal portions 312, 316. The illustrated shape of the intermediate portion 320 provides that portion with a lower torsional stiffness than either of the proximal or distal portions 312, 316, thus facilitating twisting the intermediate portion 320 so that the proximal and distal portions 312, 316 can be oriented in different, mutually orthogonal planes. In embodiments, the conductors 350 (e.g., electrical traces) connected to the second magnetic field sensor 332 can be disposed on or proximate the longitudinal axis 308 along the intermediate portion 320 so as to minimize torsional stress and strain on those conductors.

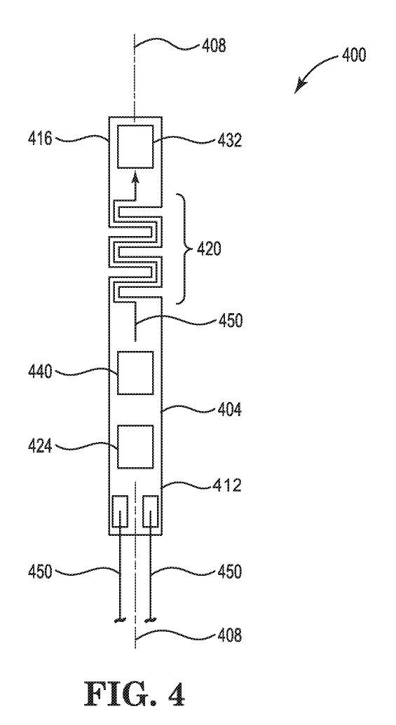

FIG. 4 is schematic view of a portion of a position sensor assembly 400 in an intermediate manufacturing state to illustrate the design according to one embodiment. Specifically, the position sensor assembly 400 includes a base member 404 defining a substantially linear longitudinal axis 408. As further shown, the base member 404 includes a proximal portion 412, a distal portion 416, and an intermediate portion 420 between the proximal and distal portions. The position sensor assembly 400 further includes a first magnetic field sensor 424 disposed on the proximal portion 412 of the base member 404, a second magnetic field sensor 432 disposed on the distal portion 416 of the base member 404, and a third magnetic field sensor 440 disposed on the proximal portion 412 at a different location than the first magnetic field sensor 424. As will be appreciated by the preceding discussion, in various embodiments, the third magnetic field sensor 440 may be instead located on the distal portion 416. Alternatively, in other embodiments, the first or second magnetic field sensor 424, 432 may be a dual-axis sensor having sensitivity in two mutually orthogonal axes, thus obviating the need for the third magnetic field sensor 440. As further shown, the position sensor assembly 400 includes a plurality of conductors 450 that operatively couple the respective magnetic field sensors to other electrical components (not shown).

In the particular embodiment illustrated, the intermediate portion 420 has a “serpentine” configuration when in the flat, untwisted state as depicted in FIG. 4. The illustrated shape of the intermediate portion 420 provides that portion with a lower torsional stiffness than either of the proximal or distal portions 412, 416, thus facilitating twisting the intermediate portion 420 so that the proximal and distal portions 412, 416 can be oriented in different, mutually orthogonal planes. In embodiments, the conductors 450 (e.g., electrical traces) connected to the second magnetic field sensor 432 can be disposed on or proximate the longitudinal axis 408 adjacent the intermediate portion 420, and then follow the serpentine path of the substrate material of the intermediate portion 420 so as to minimize torsional stress and strain on those conductors when the intermediate portion is twisted into its final configuration.

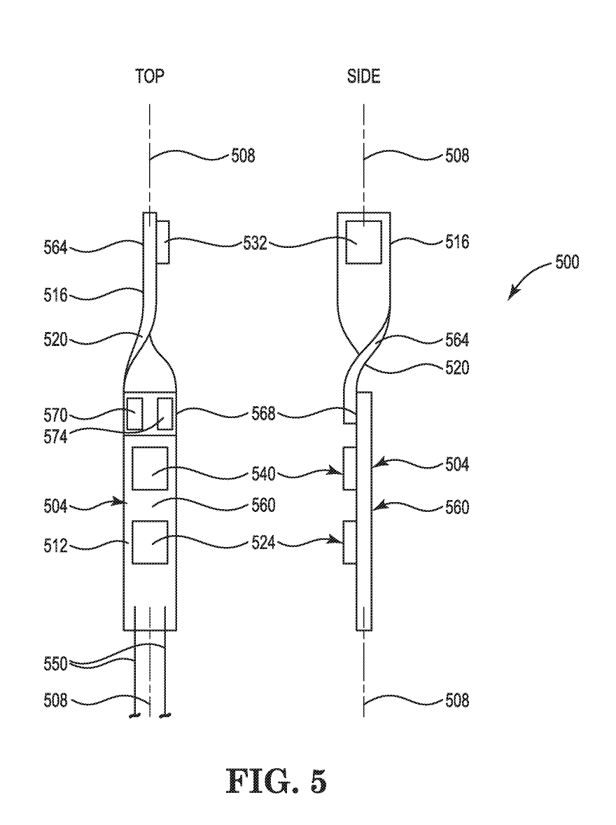

FIG. 5 illustrates top and side schematic views of a portion of a position sensor assembly 500 according to one embodiment. Specifically, the position sensor assembly 500 includes a base member 504 defining a substantially linear longitudinal axis 508. As further shown, the base member 504 includes a proximal portion 512, a distal portion 516, and an intermediate portion 520 between the proximal and distal portions. The position sensor assembly 500 further includes a first magnetic field sensor 524 disposed on the proximal portion 512 of the base member 504, a second magnetic field sensor 532 disposed on the distal portion 516 of the base member 504, and a third magnetic field sensor 540 disposed on the proximal portion 512 at a different location than the first magnetic field sensor 524. As will be appreciated by the preceding discussion, in various embodiments, the third magnetic field sensor 540 may be instead located on the distal portion 516. Alternatively, in other embodiments, the first or second magnetic field sensor 524, 532 may be a dual-axis sensor having sensitivity in two mutually orthogonal axes, thus obviating the need for the third magnetic field sensor 540. As further shown, the position sensor assembly 500 includes a plurality of conductors 550 that operatively couple the respective magnetic field sensors to other electrical components (not shown).

The base member 504 differs from those described in the previous embodiments in that it is a two-piece construction and includes a proximal base member element 560 and a distal base member element 564 mechanically and electrically coupled together at a joint 568, which can include one or more bond pads 570, 574 or other conventional structures for joining PCB components. In the illustrated embodiment the twisted intermediate portion 520 is located on the distal base member element 564. In other embodiments, the intermediate portion 520 may be located on the proximal base member element 560. The two-piece construction of the base member 504 can advantageously minimize stresses induced in the base member 504 substrate as well as in the electrical conductors 550 formed on the base member 504. In various embodiments, the intermediate portion 520 can have an hourglass or serpentine configuration as previously described.

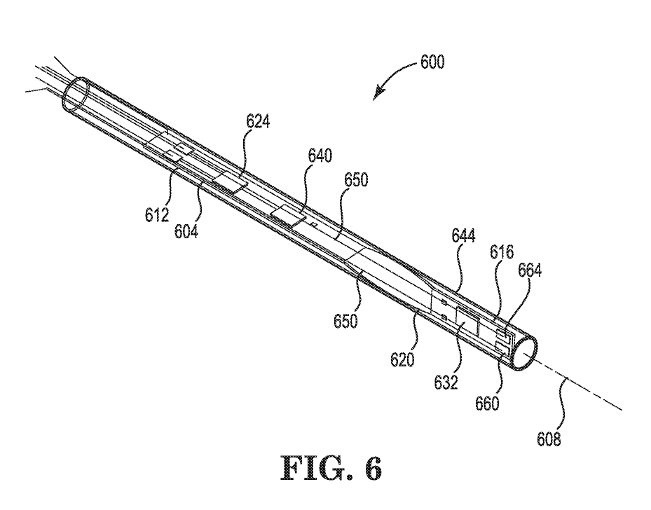

FIG. 6 is schematic view of a position sensor assembly 600 according to another embodiment. The position sensor assembly 600 is in many respect similar or identical to the position sensor assembly 200 described above, and includes a base member 604 defining a longitudinal axis 608, a proximal portion 612, a distal portion 616 and a twisted intermediate portion 620 therebetween. The position sensor assembly 600 further includes a plurality of magnetic field sensors 624, 632 and 640 disposed on the base member 604 in the manner described above in connection with other embodiments, an encapsulating element 644 (e.g., an epoxy coating), and a plurality of conductors 650 electrically coupled to the respective magnetic field sensors 624, 632 and 640.

The position sensor assembly 600 differs from the previously described embodiments in that the conductors 650 constitute lead wires that are structurally separated from (i.e., not formed on) the base member 604. In particular, the conductors 650 connected to the magnetic field sensor 632 located on the distal portion 616 of the base member 604 are carried by the encapsulating element 644 at least across the intermediate portion 620, to bond pads 660, 664 located at the distal tip of the base member 604 and electrically coupled to the magnetic field sensor 632 via short electrical traces (not shown) formed on the distal portion 616. In this configuration, the conductors 650 extending across the intermediate portion 620 are not exposed to the torsional stresses imposed on the intermediate portion 620 during the manufacturing step of forming the twist in the intermediate portion 620.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

We claim:

1. A position sensor assembly comprising:
a base member having a substantially linear longitudinal axis, a proximal portion oriented in a first plane along the longitudinal axis, a distal portion oriented in a second plane along the longitudinal axis, the second plane being substantially orthogonal to the first plane, and an intermediate portion extending between the proximal and distal portions in a twisted configuration to operate as a transition between the proximal and distal portions, wherein the base member includes a first base member element defining the proximal portion, and a second base member element defining the distal portion, and further wherein the first and second base member elements are mechanically and electrically coupled together at a joint;
a first magnetic field sensor disposed on the proximal portion of base member, the first magnetic field sensor including a first magnetic field sensing element, the first magnetic field sensor oriented on the proximal portion of the base member so that the first magnetic field sensing element has a sensitivity to a component of the multi-dimensional magnetic field along a first axis; and
a second magnetic field sensor disposed on the distal portion of base member, the second magnetic field sensor including a second magnetic field sensing element, the second magnetic field sensor oriented on the distal portion of the base member so that the second magnetic field sensing element has a sensitivity to a component of the multi-dimensional magnetic field along a second axis.

2. The position sensor assembly of claim 1, further comprising a third magnetic field sensor disposed on the proximal portion of the base member, the third magnetic field sensor including a third magnetic field sensing element, the third magnetic field sensor oriented on the proximal portion of the base member so that the third magnetic field sensing element has a sensitivity to a component of the multi-dimensional magnetic field along a third axis.

3. The position sensor assembly of claim 1, further comprising a third magnetic field sensor disposed on the distal portion of the base member, the third magnetic field sensor including a third magnetic field sensing element, the third magnetic field sensor oriented on the distal portion of the base member so that the third magnetic field sensing element has a sensitivity to a component of the multi-dimensional magnetic field along a third axis.

4. The position sensor assembly of claim 1, wherein the first magnetic field sensor includes a third magnetic field sensing element having a sensitivity to a component of the multi-dimensional magnetic field along a third axis.

5. The position sensor assembly of claim 1, wherein the second magnetic field sensor includes a third magnetic field sensing element having a sensitivity to a component of the multi-dimensional magnetic field along a third axis.

6. The position sensor assembly of claim 1, further comprising an encapsulation element hermetically encapsulating the base member and the magnetic field sensors.

7. The position sensor assembly of claim 1, wherein the intermediate portion of the base member is twisted about an axis of rotation corresponding to the longitudinal axis.

8. The position sensor assembly of claim 1, wherein the intermediate portion has a length along the longitudinal axis, and wherein a middle region of the intermediate portion is narrower than opposing end regions of the intermediate portion, so that the intermediate portion has an hourglass shape.

9. The position sensor assembly of claim 1, wherein the first base member element further defines the intermediate portion.

10. The position sensor assembly of claim 1, wherein the second base member element further defines the intermediate portion.

11. The position sensor assembly of claim 1, wherein the base member is a printed circuit board including a plurality of electrical traces operatively coupled to respective ones of the magnetic field sensors.

12. The position sensor assembly of claim 1, wherein one or more of the electrical traces is positioned proximate the longitudinal axis along the intermediate portion.

13. A medical probe comprising:
a flexible catheter body having a distal end;
an active element coupled to the distal end of the flexible catheter body; and
a position sensor assembly carried by the catheter body proximate the active element and including:
a base member having a substantially linear longitudinal axis, a proximal portion oriented in a first plane along the longitudinal axis, a distal portion oriented in a second plane along the longitudinal axis, the second plane being substantially orthogonal to the first plane, and an intermediate portion extending between the proximal and distal portions in a twisted configuration to operate as a transition between the proximal and distal portions, wherein the base member includes a first base member element defining the proximal portion, and a second base member element defining the distal portion, and further wherein the first and second base member elements are mechanically and electrically coupled together at a joint;
a first magnetic field sensor disposed on the proximal portion of base member, the first magnetic field sensor including a first magnetic field sensing element, the first magnetic field sensor oriented on the proximal portion of the base member so that the first magnetic field sensing element has a sensitivity to a component of the multi-dimensional magnetic field along a first axis; and
a second magnetic field sensor disposed on the distal portion of base member, the second magnetic field sensor including a second magnetic field sensing element, the second magnetic field sensor oriented on the distal portion of the base member so that the second magnetic field sensing element has a sensitivity to a component of the multi-dimensional magnetic field along a second axis.

14. The medical probe of claim 13, wherein the active element is a multiple-electrode mapping element.

15. The medical probe of claim 13, wherein the active element is an ablation element.

16. The medical probe of claim 13, wherein the ablation element is a radiofrequency ablation electrode or a cryoablation element.

17. The medical probe of claim 13, wherein the position sensor assembly further includes a third magnetic field sensor disposed on the proximal portion or the distal portion of the base member, the third magnetic field sensor including a third magnetic field sensing element, the third magnetic field sensor oriented so that the third magnetic field sensing element has a sensitivity to a component of the multi-dimensional magnetic field along a third axis.

18. A medical system comprising:
a medical probe comprising:
a flexible catheter body having a distal end;
an active element coupled to the distal end of the flexible catheter body; and
a position sensor assembly carried by the catheter body proximate the active element and including:
a base member having a substantially linear longitudinal axis, a proximal portion oriented in a first plane along the longitudinal axis, a distal portion oriented in a second plane along the longitudinal axis, the second plane being substantially orthogonal to the first plane, and an intermediate portion extending between the proximal and distal portions in a twisted configuration to operate as a transition between the proximal and distal portions, wherein the base member includes a first base member element defining the proximal portion, and a second base member element defining the distal portion, and further wherein the first and second base member elements are mechanically and electrically coupled together at a joint;
a first magnetic field sensor disposed on the proximal portion of base member, the first magnetic field sensor including a first magnetic field sensing element, the first magnetic field sensor oriented on the proximal portion of the base member so that the first magnetic field sensing element has a sensitivity to a component of the multi-dimensional magnetic field along a first axis; and
a second magnetic field sensor disposed on the distal portion of base member, the second magnetic field sensor including a second magnetic field sensing element, the second magnetic field sensor oriented on the distal portion of the base member so that the second magnetic field sensing element has a sensitivity to a component of the multi-dimensional magnetic field along a second axis;
a magnetic field generator configured to generate a multi-dimensional magnetic field in a volume including the medical probe and a patient; and
a processor operable to receive outputs from the magnetic field sensors to determine a position of the position sensor assembly within the volume.

19. The medical system of claim 18, wherein the position sensor assembly further includes a third magnetic field sensor disposed on the proximal portion or the distal portion of the base member, the third magnetic field sensor including a third magnetic field sensing element, the third magnetic field sensor oriented so that the third magnetic field sensing element has a sensitivity to a component of the multi-dimensional magnetic field along a third axis.

20. The medical system of claim 18, wherein one of the first and second magnetic field sensors includes a third magnetic field sensing element having a sensitivity to a component of the multi-dimensional magnetic field along a third axis
